(19) United States
(12) Patent Application Publication
(10) Pub. No.: US 2001/0016706 A1
(43) Pub. Date: Aug. 23, 2001

(54) PERISTALTIC PUMP

(76) Inventors: KURT D. LEUKANECH, LAGUNA NIGUEL, CA (US); MICHAEL D. MORGAN, COSTA MESA, CA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 09/387,034

(22) Filed: Aug. 31, 1999

(51) Int. Cl. A61M 1/00; A61M 37/00

(52) U.S. Cl. 604/140; 604/153

(57) ABSTRACT

A peristaltic pump wherein the pumping mechanism is enclosed in a vacuum chamber. Placement of the pumping mechanism within a vacuum chamber decreases the pressure differential between the inside and the outside of the pump channel, thereby minimizing changes in trapped fluid volume.

### BACKGROUND OF THE INVENTION

The present invention relates generally to peristaltic pumps and more specifically to peristaltic pumps used in ophthalmic surgical equipment.

Peristaltic pumps work by compressing or squeezing a length of flexible tubing (sometimes between a fixed race) using a rotating roller head. As the roller head rotates, the rollers stretch and pinch off a portion of the tubing and push any fluid trapped in the tubing between the roller in the direction of rotation. While it is difficult to achieve high vacuum levels with a peristaltic pump, peristaltic pumps are widely used in medical applications because of their predictable, constant flow properties.

Many factors influence the efficiency of peristaltic pumps, for example, pump motor torque, pump speed, pump tube flexibility and vacuum levels. Here, efficiency refers to the volume flow rate of a given pump and its relationship to the translational velocity of the pinching forces. The efficiency of a peristaltic pump is also dependent on the compliance or memory of the elastic material used to make the pump tubing. Some compliance in the pump tubing is required to assure that the tubing expands and returns to its undisturbed state after the translating force imparted by the rollers in the pump roller head have passed.

One disadvantage to peristaltic pumps is that for a given translation velocity (S), the average flow through the pump is adversely affected by a decrease in fluid pressure (P₁, vacuum) at the input end of the pump. This decrease in average flow results from a decrease in trapped volume (V) within the pump tubing caused by a gradual collapse of the tubing with decreasing P₁, (increasing gauge vacuum level). At very low P₁ (very high vacuum levels) the pump tubing is completely collapsed, making trapped volume V and the corresponding pump output zero.

Accordingly, a need continues to exist for a peristaltic pump with increased pumping efficiency, particularly at high vacuum levels.

### BRIEF SUMMARY OF THE INVENTION

The present invention improves upon prior art peristaltic pumps by providing a peristaltic pump wherein the pumping mechanism is enclosed in a vacuum chamber. Placement of the pumping mechanism within a vacuum chamber decreases the pressure differential between the inside and the outside of the pump tubing, thereby minimizing changes in trapped fluid volume.

Accordingly, one objective of the present invention is to provide a high efficiency peristaltic pump.

Another objective of the present invention is to provide a means for controlling the maximum achievable vacuum of a peristaltic pump.

Yet another objective of the present invention is to decrease the reliance of peristaltic pump efficiency on the compliance of the pump tubing.

These and other advantages and objectives of the present invention will become apparent from the detailed description, drawings and claims that follow.

### BRIEF DESCRIPTION OF THE DRAWINGS

FIG. 1 is a schematic representation of prior art peristaltic pumping mechanisms.

FIG. 2 is a schematic representation of the peristaltic pump mechanism of the present invention.

FIG. 3 is a partial elevational view of the peristaltic pump mechanism of the present invention.

FIG. 4 is a partial cross-sectional view of the peristaltic pump mechanism of the present invention taken at line 4-4 in FIG. 3.

FIG. 5 is a schematic representation of the operation of a peristaltic pump.

FIG. 6 is a graphic representation comparing the performance of the prior art with the present invention.

### DETAILED DESCRIPTION OF THE INVENTION

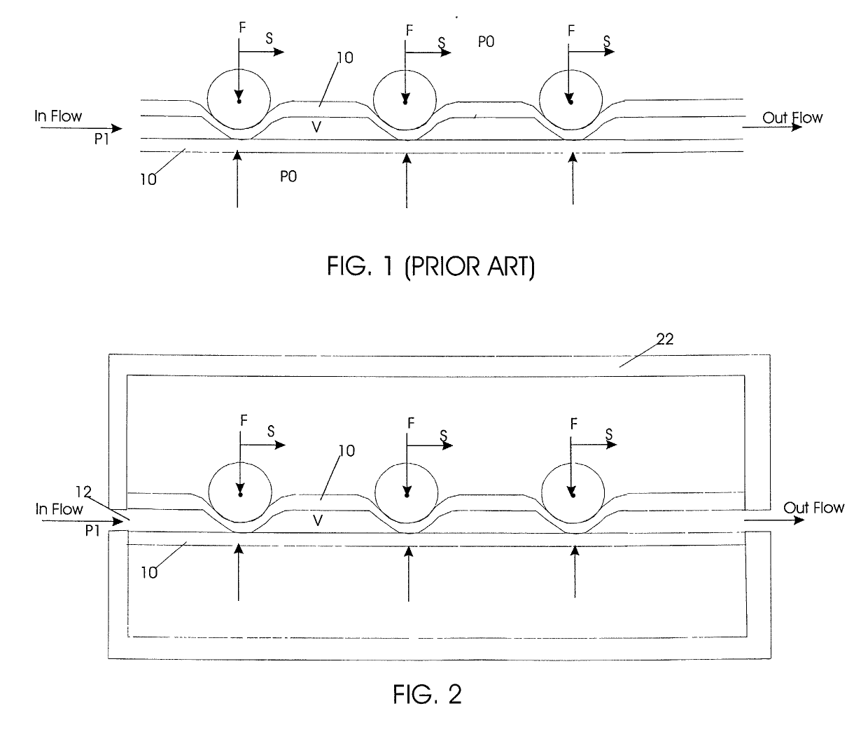

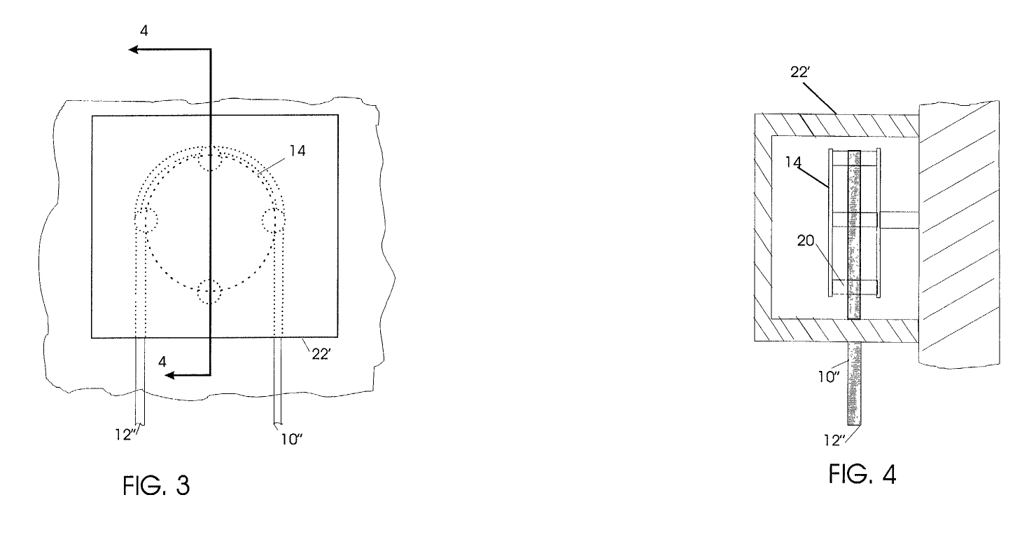

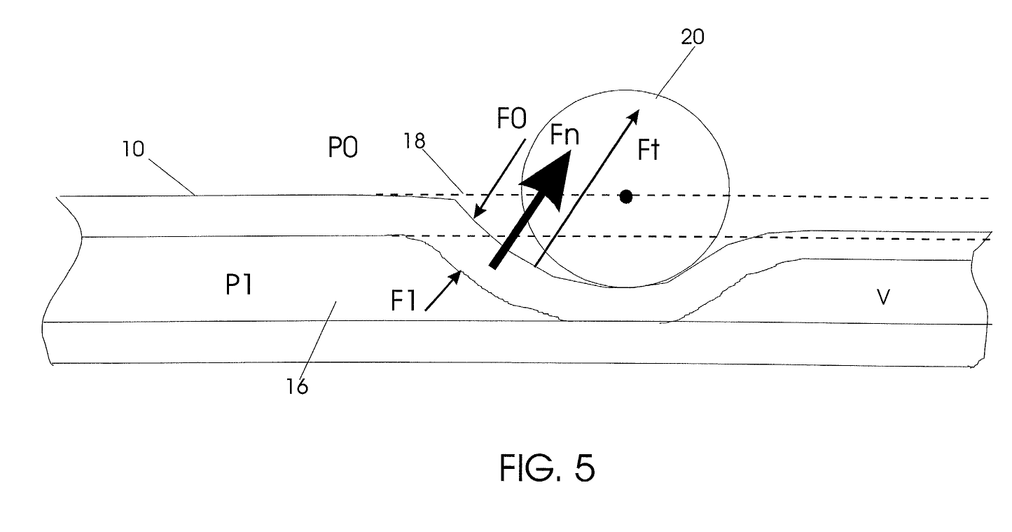

For purposes of the present invention, the term peristaltic pump mean any type of pump using peristalsis to move fluid. As best seen in FIG. 1, prior art peristaltic pumps operate at barometric external conditions. The pressure surrounding pump tube or channel 10 is barometric pressure P₀. Fluid flow within pump channel 10 is caused by a sequential, rolling series of pinching forces F along the length of channel 10. As seen in FIGS. 3, 4 and 5, these pinching forces are generally supplied by rotating head 14 or other device having a series of spaced rollers 20. Each of the pinching forces creates a small trapped volume V of fluid that is propelled along channel 10 by the sequential nature of forces F. As shown in FIG. 5, fluid is drawn into pump channel 10 by the return of pump channel 10 to its expanded, unpinched state 18 after pump roller 20 has passed by. While the average flow rate is generally proportional to the speed S of rotating head 14, average flow rate is adversely affected by a decrease in fluid pressure P₁ within pump channel 10. This decrease in average fluid flow is due to a decrease in volume V resulting from the gradual collapse of pump channel 10 with decreasing pressure P₁.

Also shown in FIG. 5 are the forces involved in producing flow. Force Fₜ is the return force of compliant pump channel 10. Force F₀ is the force due to the pressure P₀ surrounding pump channel 10. Force F₁ is the force due to the pressure P₁ within pump channel 10. The resultant force Fₙ is responsible for performing the work of drawing the fluid through pump channel 10 and is the vector sum of all of the forces involved: Fₙ=Fₜ+(F₁−F₀).

For the existing art, F₀ is due to P₀ being equal to barometric pressure, and internal pressure P₁ work against the ability of the system to draw fluid because of the low levels of F₁. At some point, P₁ can reach a high level of vacuum (very low levels of F₁) such that Fₙ reaches zero. At this point, pump channel 10 remains collapsed and average flow is zero. The level of P₁ at which the average flow is zero is at the maximum achievable vacuum of the pump Vₘₐₓ.

The above discussion demonstrates that if P₀ is decreased relative to barometric pressure, the average flow at high levels of vacuum (P₁) will be improved and the maximum achievable vacuum Vₘₐₓ will also be improved. Conversely, Vₘₐₓ can now be controlled to any desired level by controlling P₀. In addition, one skilled in the art will recognize that pressures greater than barometric pressure may also be used to lower Vₘₐₓ.

Operation of a peristaltic pump at high vacuum levels places significant design constraints on pump channel 10. These constraints add to the cost of pump channel 10 as well as limit the selection of materials capable of meeting the design requirements for pump channel 10. The present invention allows for a relaxation of the design constraints for pump channel 10, and new types of materials for pump channel 10 which are compressible yet inelastic to expansion. For example, polyester film (e.g. MYLAR®) or other suitable materials can be used where Fₜ is zero so Fₙ=F₁−F₀.

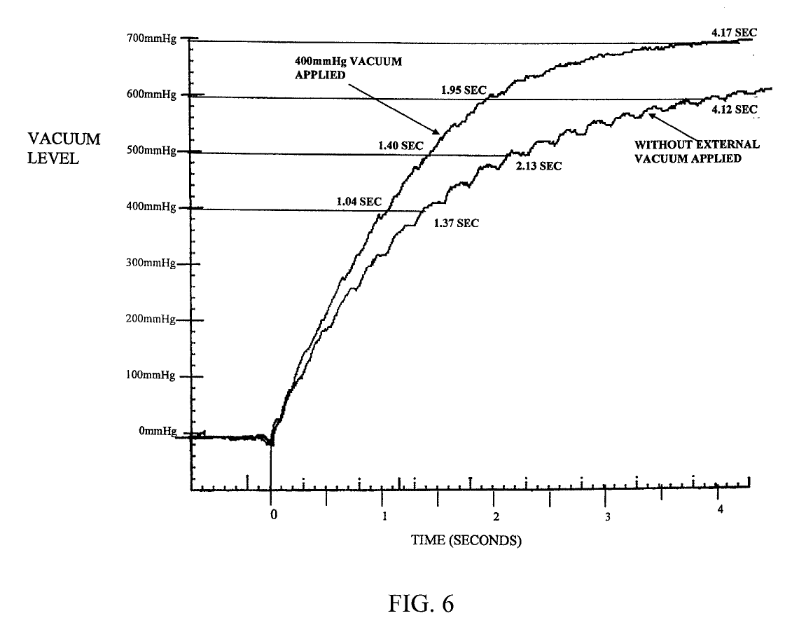

As illustrated in FIGS. 2, 3 and 4, the inventors have found that by placing the peristaltic pump mechanism inside pressure or vacuum chamber 22 or 22′, the degrading effects of vacuum P₁ inside channel 10 or 10″ can be reduced or eliminated. See FIG. 6. By introducing a vacuum inside chamber 22 or 22′ relative to P₀, the collapsing force on channel 10 or 10″ caused by P₁ can be negated, and any reduction in trapped volume v caused by partial collapse of channel 10 and 10″ as a result of P₁ can also be reduced. One skilled in the art will recognize that the present invention is not limited to peristaltic pumps using a roller head and a pump tube but also encompasses any type pump using peristalsis, such as linear peristaltic pumps.

As seen in FIG. 6, to test the effectiveness of the present invention, the inventor constructed a system where pump channel 10 or 10″ was placed within a vacuum chamber 22 or 22′. The internal pressure within chamber 22 or 22′ was varied from barometric to 400 mmHg below barometric pressure. The two graphs shown in FIG. 6 demonstrate the time necessary for the pump to evacuate a constant, fluid filled volume for a pump when pump channel 10 or 10″ is exposed to barometric pressure and for a pump when pump channel 10″ or 10″ is exposed to 400 mmHg below barometric pressure. As can be seen, there is a significant decrease in evacuation time for the system when pump channel 10 or 10″ is exposed to 400 mmHg below barometric pressure, as well as an increase for Vₘₐₓ. One skilled in the art will recognize that lower pressures, as low as 760 mmHg below barometric pressure may also be used. In addition, one skilled in the art will recognize that pressures greater than barometric pressure may also be used.

This description is given for purposes of illustration and explanation. It will be apparent to those skilled in the relevant art that modifications may be made to the invention as herein described without departing from its scope or spirit.

I claim:

1. A peristaltic pump, comprising:
a) a pressure container;
a) a peristaltic pump channel; and
b) a means for applying pinching forces to the peristaltic pump channel, wherein the peristaltic pump channel and the means for applying pinching forces to the pump channel are contained within the pressure container.

2. The peristaltic pump of claim 1 wherein the internal pressure of the container is maintained at between 0 mmHg and 760 mmHg below barometric pressure.

3. The peristaltic pump of claim 1 wherein the internal pressure of the container is maintained at a pressure greater than barometric pressure.

4. The peristaltic pump of claim 1 wherein the pump tube comprises a polyester film.

5. A method of operating a peristaltic pump, comprising the steps of:
a) placing a peristaltic pump channel within a pressure container;
b) reducing the pressure in the pressure container to below barometric pressure; and
c) applying pinching forces to the peristaltic pump channel so as to draw fluid through the pump channel.

6. A method of varying the vacuum achievable by a peristaltic pump, the method comprising the steps of:
a) placing a peristaltic pump channel within a pressure container; and
b) variably reducing the pressure in the pressure container to at or below barometric pressure while applying pinching forces to the peristaltic pump channel so as to draw fluid through the pump channel.
